(19) United States
(12) Patent Application Publication
(10) Pub. No.: US 2022/0054552 A1
(43) Pub. Date: Feb. 24, 2022

(54) Cellular pharmaceutical product containing quantified doubling level stem cells and a method for producing it

(71) Applicant: Jackson Tsung-Kui Liu, Kaohsiung (TW)

(72) Inventor: Jackson Tsung-Kui Liu, Kaohsiung (TW)

(21) Appl. No.: 16/996,924

(22) Filed: Aug. 19, 2020

(51) Int. Cl. A61K 35/28

(52) U.S. Cl. CPC A61K 35/28

(57) ABSTRACT

The present invention relates to a pharmaceutical cellular composition containing quantified doubling level nucleated cells and a method for producing it.

### FIELD OF THE INVENTION

The present invention relates to a cellular pharmaceutical composition containing stem cell rich nucleated cells of measured doubling level, calculated total proliferation cell number and a method for producing it.

### BACKGROUND OF THE INVENTION

The art and knowledge of stem cells have become more sophisticated over the years and many more types of progenitors of cell are identified and described, for example, umbilical cord blood now known not only to contain only hematopoeitic stem cells, but may contain many non-hematopoeitic cells too, such as endothelial colony forming cells, unrestricted somatic stem cells, mesenchymal stromal cells, very small embryonic-like stem cells, multilineage progenitor cells, neuronal progenitor cells.

Most patent applications focus on the morphological characterization (e.g. mesenchymal, epithelial), source origin (adipose, placental, cord, blood etc) and surface antibody affinity (CD markers) of the cells yet little has given credit to one of the other most important features of stem cells: their ability to undergo mitosis and proliferate to more cells. It is well known partially differentiated stem cells, mesenchymal or epithelial, will further expand to their lineage but little has invest in how much further they will proliferate.

### DISCLOSURE OF THE INVENTION

Stem cells are undifferentiated biological cells that can differentiate into specialized cells and can divide to produce more stem cells. Accessible sources of stem cells include fat tissue, bone marrow, blood, umbilical cord blood, umbilical cord and placenta. Stem cells (totipotent) lose its ability to divide indefinitely early if not maintained in special environment and starts to display Hayflick's phenomenon.

It is more than rational to expected different number of remaining doubling level will affect culture, experiment or treatment results, e.g. cells with the ability of double level 50 will certainly do better in comparison to cells with ability to doubling of 5 times. It is evidently observed in the clinical transplantation using nucleated stem cells of dosage of almost 10 times more when using cells source from adult bone marrow in comparison to using source from umbilical cord blood. It has also been observed in umbilical cord blood transplantation, the number of nucleated cells transplanted being the strongest correlation to the success of treatment besides the number of HLA matching.

The present invention specify a product with a quantified nucleated cells from stem cell rich source, sample tested doubling level, potential total cell number the product can proliferate to based on sample culturing and method for producing it.

### SUMMARY OF THE INVENTION

The present invention relates to a cellular pharmaceutical composition containing stem cell rich nucleated cells of measured doubling level, projected total proliferation cell number and a method for producing it.

### DETAILED DESCRIPTION OF THE INVENTION

The present invention relates to a cellular pharmaceutical composition containing stem cell rich nucleated cells of sample measured doubling level, calculated total proliferation cell number and a method for producing it.

Cells from a single source after initial collection is counted for cell concentration, volume and total nucleated cell number.

It is then centrifuged and processed to the concentration of 1.1×10⁸ cell/ml, total volume of cell suspension X ml is obtained.

Calculate number of bottles units, n, from number of 3 ml(+/−5%) per bottle can be obtained from the volume X; remaining volume, r, needed to be >0.1 ml after 3 ml +/−5% bottling adjustment. If r can not be 0.1 ml after best effort adjustment, a bottle from n is sacrificed to make r>0.1 ml.

Remove r from X. Add 10% cryo-protectant to remaining (X-r) and divide equally, (3 ml(+/−5%)+10%) in n bottles and preserve cryogenically . If freshly collected cells is less than 3.3×10⁸ cells, bottling step is skipped and goes to culturing step directly (n=0).

Remaining r is cultured in mixed cell type culture medium. Passage, time in hour and cell number doubling(d) is recorded.

Passage is continued until cells reached over 5 doublings, i.e. >32 times (>3.52×10⁸) cells, and then bottling processed as previously described with centrifugation and processing to concentration of 1.1×10⁸ cell/ml, then 3 ml bottle(s) are cryogenically preserved and remaining volume of >0.1 ml is cultured in mixed type medium.

The cycle steps were repeated until the cells stopped proliferating and total doubling level is recorded, nPDL, by number of times the steps repeated times 5 and add dividing logarithm of the fold increase value obtained at the last cycle of the passage by the logarithm of 2.

The doubling level ability of the first batch bottle product has the nPDL, the second batched is n−5 PDL, third is n−10 PDL etc., for example, if 38 doubling was achieved for this single source cells, first batch bottle products are sample tested to have the ability of 38 doubling potential, second batch will have 33, third batch 28 and so goes on.

The bottle products can be increased to more number of bottle products at sacrifice of the potential doubling level. For example, if it is tested the potential doubling level of the specific source to be 38, and 30 doubling level is specified for the product, then batches with 38 doubling potential can be thawed and cultured for 8 doubling (38−30=8) and produce 256 times more bottle products.

Every bottles are thus sampled tested for doubling level when produced from the steps. Bottles are also calculated for its potential of total cell proliferation with the doubling level, e.g. doubling level of 38 with 3×10⁸ cells can potentiaally proliferate to total cell of =3.3×10⁸×2³⁸=9.07×10¹⁹ cells.

### SUMMARY OF THE INVENTION

The present invention relates to a cellular pharmaceutical composition containing stem cell rich nucleated cells of measured doubling level, projected total proliferation cell number and a method for producing it.

### REFERENCES CITED

AU 2006332678 B2 2006 Labazzo et al.

EP 2 210 608 B1 2008 Shirono et al.

### LITERATURE CITED

J Y Xu et al. (2016). Stem cell transplantation dose in patients with acute myocardial infarction: A meta-analysis. Chronic Diseases and Translational Medicine (2016) 1e10.

Matsumoto T, Mugishima H (2009). Non-Hematopoi etic Stem Cells in Umbilical Cord Blood. Int J Stem Cells. 2009 November; 2(2): 83-89.

Hayflick L, Moorhead PS (1961). “The serial cultivation of human diploid cell strains”. Exp Cell Res. 25 (3): 585-621.

1. A pharmaceutical composition which comprises 3×10⁸ nucleated cells, from stem cells rich source, in 3 ml volume, sample tested for doubling potential level and calculated projection of its total cell proliferation potential.

2. Potential doubling level/number of products in claim 1 can be specified (to a lower doubling level/number).

3. Time for doubling, potential doubling level and potential proliferation cell number are applied in clinical treatment for treatment on set time, effectiveness and duration correlation and estimation.
